SEQUENCE LISTING (15 sequences)

SEQ ID NO: 1 - protein, 469 aa, Panax L.
MDLFISSQLLLLLVFCLFLFWNFKPSSQNKLPPGKTGWPIIGETLEFISCGQKGNPEKFVTQRMNKYSPDVFTTSLAGEKMVVFCGASGNKFIFSNENKLVVSWWPPAISKILTATIPSVEKSKALRSLIVEFLKPEALHKFISVMDRTTRQHFEDKWNGSTEVKAFAMSESLTFELACWLLFSINDPVQVQKLSHLFEKVKAGLLSLPLNFPGTAFNRGIKAANLIRKELSVVIKQRRSDKLQTRKDLLSHVMLSNGEGEKFFSEMDIADVVLNLLIASHDTTSSAMGSVVYFLADHPHIYAKVLTEQMEIAKSKGAGELLSWEDIKRMKYSRNVINEAMRLVPPSQGGFKVVTSKFSYANFIIPKGWKIFWSVYSTHKDPKYFKNPEEFDPSRFEGDGPMPFTFIPFGGGPRMCPGSEFARLEVLIFMHHLVTNFKWEKVFPNEKIIYTPFPFPENGLPIRLSPCTL

SEQ ID NO: 2 - DNA, 20 bp, Artificial Sequence
  Primer
atggatttgtttatttcttc

SEQ ID NO: 3 - DNA, 20 bp, Artificial Sequence
  Primer
ttacaatgtacatggagaca

SEQ ID NO: 4 - DNA, 20 bp, Artificial Sequence
  Primer
cccaaagctaagagtcccat

SEQ ID NO: 5 - DNA, 59 bp, Artificial Sequence
  Primer
gtagaaacattttgaagctatggtgtgtgggggatcactctgctcttgaatggcgacag

SEQ ID NO: 6 - DNA, 59 bp, Artificial Sequence
  Primer
aacactggggcaataggctgtcgccattcaagagcagagtgatcccccacacaccatag

SEQ ID NO: 7 - DNA, 59 bp, Artificial Sequence
  Primer
aacaataacaattgtgaagaaataaacaaatccattttgtaattaaaacttagattaga

SEQ ID NO: 8 - DNA, 59 bp, Artificial Sequence
  Primer
gaaagcatagcaatctaatctaagttttaattacaaaatggatttgtttatttcttcac

SEQ ID NO: 9 - DNA, 59 bp, Artificial Sequence
  Primer
agtgtctcccgtcttctgtctaatgatgatgatgatgatgcaatgtacatggagacaat

SEQ ID NO: 10 - DNA, 59 bp, Artificial Sequence
  Primer
attgtctccatgtacattgcatcatcatcatcatcattagacagaagacgggagacact

SEQ ID NO: 11 - DNA, 59 bp, Artificial Sequence
  Primer
ctgtcgattcgatactaacgccgccatccagtgtcgaattttcaacatcgtattttccg

SEQ ID NO: 12 - DNA, 59 bp, Artificial Sequence
  Primer
cattatgcaacgcttcggaaaatacgatgttgaaaattcgacactggatggcggcgtta

SEQ ID NO: 13 - DNA, 59 bp, Artificial Sequence
  Primer
aattcaaaaaaaaaaagcgaatcttcccatgcctgttcagcgacatggaggcccagaat

SEQ ID NO: 14 - DNA, 59 bp, Artificial Sequence
  Primer
agactgtcaaggagggtattctgggcctccatgtcgctgaacaggcatgggaagattcg

SEQ ID NO: 15 - DNA, 22 bp, Artificial Sequence
  Primer
tctggtgaggatttacggtatg

(19) United States
(12) Patent Application Publication
(10) Pub. No.: US 2024/0035062 A1
(43) Pub. Date: Feb. 1, 2024

(54) Cytochrome P450 Mutant Protein and Use Thereof

(71) Applicant: GsynBioT (Shanghai) Co., Ltd., Shanghai (CN)

(72) Inventors: Zhihua Zhou, Shanghai (CN); Pingping Wang, Shanghai (CN); Xing Yan, Shanghai (CN)

(21) Appl. No.: 18/037,339

(22) PCT Filed: Nov. 16, 2021
(86) PCT No.: PCT/CN2021/130830

§ 371 Date: Oct. 19, 2023

(30) Foreign Application Priority Data
Nov. 17, 2020 (CN) 202011285843.2

(51) Int. Cl. C12P 33/06; C12N 9/02

(52) U.S. Cl. CPC C12P 33/06; C12N 9/0071; C12Y 114/14001

(57) ABSTRACT

Provided are a cytochrome P450 (CYP716A53v2) mutant protein and the use thereof. The mutation is mutated at a site selected from the group consisting of F167V, T451A, I117S and L208C or a combination thereof on the basis of a wild-type CYP716A53v2 protein. The enzyme catalytic activity of the CYP716A53v2 mutant obtained after mutation is improved, and the yield of panaxatriol can be improved.

### RELATED APPLICATIONS

This application is a National Stage Application under 35 U.S.C. 371 of expired PCT application PCT/CN2021/130830 designating the United States and filed Nov. 16, 2021; which claims the benefit of CN application number 202011285843.2 and filed Nov. 17, 2020, each of which are hereby incorporated by reference in their entireties.

### SEQUENCE LISTING

The instant application contains a Sequence Listing which has been submitted electronically in ANSI format and is hereby incorporated by reference in its entirety. Said ANSI copy, created on Oct. 17, 2023, is named “208308_sequence listing” and is 7 KB in size.

### TECHNICAL FIELD

The disclosure relates to fields of biotechnology and natural product medicine, in particular, the disclosure relates to cytochrome P450 mutant protein (CYP716A53v2) and use thereof.

### BACKGROUND

Ginsenosides are the main active substances in Araliaceae Panax plants (such as ginseng, Panax notoginseng, Panax quinquefolius L., etc.). In recent years, some ginsenosides have also been found in Cucurbitaceae plant Gynostemma. At present, scientists at home and abroad have isolated at least more than 100 ginsenosides from ginseng, Gynostemma pentaphyllum and other plants, and the content of these saponins in ginseng varies greatly. Some of these triterpenoid saponins with significant curative effect are very low in natural total saponins (also known as rare saponins). These saponins are very expensive due to high costs of extraction. Currently, a variety of saponins have been used in medicines and health products. For example, the drug Shenyi capsule with ginsenoside Rg3 monomer as the main component can improve the symptoms of qi deficiency in patients with cancer and improve immune functions; Rui Desheng capsule with a mixture of 16 rare ginsenosides such as ginsenoside Rh1 as the main component can inhibit angiogenesis in tumor sites, promote apoptosis of cancer cells and reduce the resistance to chemotherapy.

Because rare ginsenosides often have unique biological activities or more significant therapeutic effects, traditionally prepared rare ginsenosides are prepared from a large number of saponins extracted from ginseng or Panax notoginseng by chemical hydrolysis, enzymatic hydrolysis and microbial hydrolysis. Since wild ginseng resources have been basically exhausted, total ginsenoside resources are mainly derived from the artificial cultivation of ginseng or Panax notoginseng recently. However, artificial cultivation of ginseng needs a long growth cycle (generally more than 5-7 years), with limitation of areas, and often affected by diseases and pests, thereby a large amount of pesticides are needed to apply. Besides, the artificial cultivation of ginseng or Panax notoginseng has serious continuous cropping obstacles (the ginseng or Panax notoginseng planting land needs to be fallow for more than 5-15 years to overcome the continuous cropping obstacles), so yield, quality and security of ginsenosides all face challenges. On the other hand, because there are still a large number of components in total saponins that cannot be converted into target ginsenoside monomers (such as protopanaxatriol-type saponins) and cannot be utilized, using total ginsenosides as raw materials to prepare single-component saponins not only causes waste of resources, but also increase the cost of extraction and purification.

Synthetic biology provides new opportunities for the heterologous synthesis of plant-derived natural products. Using yeast as the chassis, through the assembly and optimization of metabolic pathways, cheap monosaccharides have been fermented to synthesize artemisinic acid or dihydroartemisinic acid, and then artemisinin can be produced by one-step chemical transformation, indicating that synthetic biology has great potential for drug synthesis from natural products. Using yeast chassis cells to heterologously synthesize rare ginsenoside monomers by synthetic biological methods, raw materials are cheap monosaccharides, and preparation process is a safety-adjustable fermentation process, avoiding any external pollution (for example, the pesticides used in artificial planting of raw materials). Therefore, the preparation of rare ginsenoside monomers by synthetic biology not only shows advantages in costs, but also ensures the quality and safety of finished products Using synthetic biology to prepare sufficient amounts of various rare ginsenoside monomers with high purities for activity determination and clinical experiments promotes the development of innovative drugs for rare ginsenosides.

To synthesize protopanaxatriol-type ginsenosides with pharmaceutical activities by synthetic biology, it is necessary to analyze and reconstruct the anabolic pathway of protopanaxatriol PPT firstly. Since ginsenosides are triterpenoids, the MVA and MEP metabolic pathways in plants provide common precursors of terpenoids IPP and DMAPP, laying a foundation for the synthesis of triterpenoid precursors squalene and 2,3-epoxysqualene. In 2006, Korean and Japanese scientists cloned and identified the synthase DS that converts epoxysqualene to dammarediol from ginseng respectively (Han, J. Y., et al., Plant Cell Physiol, 2006. 47(12): p. 1653-62.; Tansakul, P., et al., FEBS Lett, 2006. 580(22): p. 5143-9), In 2011 (Han, J. Y., et al., Plant Cell Physiol, 2011. 52(12): p. 2062-73) and 2012 (Han, J. Y., et al., Plant Cell Physiol, 2012. 53(9): p. 1535-45), korean researcher Han J Y cloned and characterized key cytochrome P450s, CYP716A47 and CYP716A53v2, for the synthesis of protopanaxadiol and protopanaxatriol, from a cDNA library of ginseng, respectively. CYP716A47 can catalyze the hydroxylation of C12-position of dammarediol to form protopanaxadiol PPD, and CYP716A53v2 can catalyze the hydroxylation of C6-position of protopanaxadiol to form protopanaxatriol PPT. DS, two cytochrome P450s and the Arabidopsis-derived P450 reductase ATR2-1 were co-expressed in WAT21 yeast, and a recombinant strain capable of producing protopanaxadiol and protopanaxatriol was obtained. Further studies showed that the conversion of protopanaxadiol to protopanaxatriol catalyzed by CYP716A53v2 is a key rate-limiting step in the entire synthetic pathway.

Therefore, more researches and modifications of cytochrome P450 CYP716A53v2 are needed in this field to obtain more efficient cytochrome P450 protein elements for promoting the synthesis efficiency of ginsenoside cell factory.

### SUMMARY OF THE DISCLOSURE

The disclosure mutates and optimizes the protein coding sequence of cytochrome P450 CYP716A53v2 to obtain a new mutant sequence. By expressing the mutant sequence in cells producing protopanaxadiol can significantly increase the yield of protopanaxatriol.

In the first aspect, the disclosure provides a method for improving catalytic activities of cytochrome P450 CYP716A53v2, comprising: a mutation in the amino acid sequence of cytochrome P450 CYP716A53v2, wherein the mutation is mutated at a position selected from the group consisting of 167, 451, 117 and 208 or a combination thereof corresponding to wild-type CYP716A53v2 protein.

In a preferred example, the numbering of amino acid positions is based on the amino acid sequence shown in SEQ ID NO: 1.

In another preferred example, position 167 is mutated to Val (V), position 451 is mutated to Asn (A), position 117 is mutated to Ser (S), and position 208 is mutated to Cys (C).

In another aspect, the disclosure provides a cytochrome P450 CYP716A53v2 mutant, the mutant is: (a) a mutant protein with an amino acid mutation corresponding to wild-type CYP716A53v2 at a position selected from the group consisting of 167, 451, 117 and 208 or a combination thereof (preferably, these mutations are core amino acid mutations); (b) a protein derived from (a) and with the function of protein (a), formed by one or more amino acid residue substitution, deletion or addition of protein (a), but the residue corresponding to the position of 167, 451, 117 or 208 of wild-type CYP716A53v2 is as same as the corresponding residue of protein (a); (c) a protein derived from (a) and with the function of protein (a), with more than 80% (preferably more than 85%; more preferably more than 90%; more preferably more than 95%, for example 98%, 99%) homology to the amino acid sequence of protein (a), but the residue corresponding to the position of 167, 451, 117 or 208 of wild-type CYP716A53v2 is as same as the corresponding residue of protein (a); or, (d) a polypeptide derived from any one of the amino acid sequences of (a) to (c) with a tag or an enzyme-cleavage sequence added at N-terminus or C-terminus; or, a signal polypeptide or a secretory signal sequence fused at N-terminus.

In a preferred example, the cytochrome P450 CYP716A53v2 has significantly higher catalytic activities than the wild type.

In another preferred example, in the cytochrome P450 CYP716A53v2 mutant, position 167 is mutated to Val (V).

In another preferred example, in the cytochrome P450 CYP716A53v2 mutant, position 451 is mutated to Asn (A).

In another preferred example, in the cytochrome P450 CYP716A53v2 mutant, position 117 is mutated to Ser (S).

In another preferred example, in the cytochrome P450 CYP716A53v2 mutant, position 208 is mutated to Cys(C).

In another preferred example, corresponding to wild-type cytochrome P450 CYP716A53v2, the mutant comprises a protein selected from the group consisting of:
(1) position 117 is mutated to Ser, position 451 is mutated to Ala;
(2) position 117 is mutated to Ser, position 208 is mutated to Cys, position 167 is mutated to Val, position 451 is mutated to Ala;
(3) position 117 is mutated to Ser, position 208 is mutated to Cys;
(4) position 117 is mutated to Ser, position 208 is mutated to Cys, position 451 is mutated to Ala;
(5) position 167 is mutated to Val;
(6) position 451 is mutated to Ala;
(7) position 117 is mutated to Ser;
(8) position 208 is mutated to Cys.

In another aspect, the disclosure provides an isolated polynucleotide, wherein, the nucleic acid encodes any one of the cytochrome P450 CYP716A53v2 mutants described above.

In another aspect, the disclosure provides a vector, wherein it comprises the polynucleotide.

In a preferred example, the vector comprises expression vector, shuttle vector, integrating vector.

In another aspect, the disclosure provides a genetically engineered host cell, wherein it comprises any one of the vectors described above, or any one of the polynucleotides described above and integrated into genome.

In another preferred example, the host cell is a eukaryotic cell or a prokaryotic cell; preferably, the eukaryotic cell comprises (but not limited to): yeast cell, plant cell, fungal cell, insect cell, mold cell, mammalian cell; more preferably, the yeast cell comprises (but not limited to): cells of Saccharomyces cerevisiae or Pichia pastoris (more preferably, the yeast cell is the cell of Saccharomyces cerevisiae); more preferably, the plant cell comprises (but not limited to): ginseng cell; preferably, the prokaryotic cell comprises (but not limited to): cells of Escherichia coli, Bacillus subtilis.

In another aspect, the disclosure provides a method for preparing any one of the cytochrome P450 CYP716A53v2 mutants described above, comprising:
(i) culturing the host cell;
(ii) collecting the culture comprising the cytochrome P450 CYP716A53v2 mutant;
(iii) separating the cytochrome P450 CYP716A53v2 mutant from the culture.

In another aspect, the disclosure provides a composition for catalyzing protopanaxadiol to generate protopanaxatriol, comprising an effective amount of: any one of the cytochrome P450 CYP716A53v2 mutants described above; or, the host cell or a culture or lysate thereof; and food-acceptable or industrially acceptable carrier.

In a preferred example, the catalysis is high-efficient catalysis, with its efficiency of at least more than 10% higher than that of the wild type, preferably at least more than 20% higher, more preferably at least more than 30% higher, such as more than 40% higher, more than 50% higher, more than 60% higher.

In another aspect, the disclosure provides a use of any one of the cytochrome P450 CYP716A53v2 mutants described above for catalyzing protopanaxadiol to generate protopanaxatriol; preferably, the cytochrome P450 CYP716A53v2 mutant generates protopanaxatriol by adding a hydroxyl group at the C6 position of protopanaxadiol.

In another aspect, the disclosure provides a use of the composition for catalyzing protopanaxadiol to generate protopanaxatriol; preferably, the cytochrome P450 CYP716A53v2 mutant generates protopanaxatriol by adding a hydroxyl group at the C6 position of protopanaxadiol.

In another aspect, the disclosure provides a method for catalyzing protopanaxadiol to generate protopanaxatriol, wherein the method comprises: utilizing any one of the cytochrome P450 CYP716A53v2 mutants described above or the composition to treat protopanaxadiol; preferably, the cytochrome P450 CYP716A53v2 mutant generates protopanaxatriol by adding a hydroxyl group at the C6 position of protopanaxadiol.

In another aspect, the disclosure provides a kit for catalyzing protopanaxadiol to generate protopanaxatriol, comprising: the cytochrome P450 CYP716A53v2 mutant or the combination of mutants thereof; the host cell; or the composition.

Other aspects of the present disclosure will be apparent to those skilled in the art based on the invention herein.

### DESCRIPTION OF FIGURES

FIG. 1 shows PPT yield of integrated mutant CYP716A53v2 in chassis cells of Saccharomyces cerevisiae.

### DETAILED DESCRIPTION

After in-depth researches, the inventors established a large number of CYP716A53v2 mutants, carried out functional research of the mutants, determined the amino acid sites related to the catalytic activity of the enzyme, and obtained mutants with significantly improved catalytic activity of the enzyme through site-directed transformation.

### Mutants of the Present Disclosure and Encoding Nucleic Acids Thereof

The inventors established a mutant library of cytochrome P450 (CYP716A53v2) using Saccharomyces cerevisiae chassis cells ZW that synthesize protopanaxadiol PPD: by transforming randomly mutated CYP716A53v2 gene into Saccharomyces cerevisiae chassis cells ZW, a yeast mutant library of CYP716A53v2 with a single-copy insertion in yeast genome and synthesis of protopanaxatriol PPT was established. Based on PPT yield of the strain, the present inventors identified key amino acid positions that improve the activity of CYP716A53v2. It is found in the present disclosure that some mutants obtained by modifying the key positions of cytochrome P450 (CYP716A53v2) can increase the PPT yield.

As used herein, the term “mutant (protein)”, “CYP716A53v2 mutant”, “mutant CYP716A53v2” are used interchangeably and all refer to a non-naturally occurring protein that catalyzes the production of protopanaxadiol to protopanaxatriol, and the mutant protein is the protein shown in SEQ ID NO: 1, or a protein artificially modified based on the protein shown in SEQ ID NO: 1 (comprising variants, derivatives, etc. with variations on inactive positions) wherein, the mutant comprises core amino acids related to enzyme catalytic activity, and at least one of the core amino acids is artificially modified; and the mutant of the present disclosure has enzymatic activityto catalyze the C6 hydroxylation of protopanaxadiol (PPD) to generate protopanaxatriol (PPT).

The term “core amino acid” refers to a sequence based on SEQ ID NO: 1 and having at least 80%, such as 84%, 85%, 90%, 92%, 95%, 98% homology to SEQ ID NO: 1, the corresponding position is the specific amino acid described herein, for example, corresponding to the sequence shown in SEQ ID NO: 1, the core amino acids are: amino acid at position 167 is V; amino acid at position 451 is A; amino acid at position 117 is S; amino acid at position 208 is C; amino acids at position 117 is S and position 208 is C; amino acids at position 117 is S and position 451 is A; amino acids at position 117 is S, position 208 is C and position 451 is A; amino acids at position 117 is S, position 167 is V, position 208 is C and position 451 is A.

It should be understood that the amino acid numbering in mutants of the present disclosure is based on SEQ ID NO: 1. When a specific mutant protein has 80% or more than 80% homology with the sequence shown in SEQ ID NO: 1, the amino acid numbering of the mutant protein may be a misplacement relative to the amino acid numbering of SEQ ID NO: 1, such as 1-5 positions to the N-terminus or C-terminus of the amino acids. However, by conventional sequence alignment in the art, those skilled in the art can usually understand such misplacement is within a reasonable range. Mutant proteins with 80% (for example 90%, 95%, 98%) homology, the same or similar enzymatic activities should not be due to the misplacement of the amino acid numbering, and are not within the scope of the mutant proteins in the present disclosure.

The mutant (mutant protein) of the disclosure is a synthetic or recombinant protein, that is, can be a chemically synthesized product, or can be produced by prokaryotic or eukaryotic hosts (for example, bacteria, yeast, plants) using recombinant technology. Depending on the host used in the recombinant production, the polypeptide of the present disclosure may be glycosylated or non-glycosylated. The mutant protein of the present disclosure may also include or not include the initial methionine residue.

The present disclosure also comprises fragments, derivatives and analogues of the mutant proteins. As used herein, the terms “fragment”, “derivative”, and “analogue” refer to proteins that maintain essentially the same biological function or activity as the mutant protein.

Fragments, derivatives, or analogues of the mutant protein of the present disclosure may be (i) a mutant protein having one or more conservative or nonconservative amino acid residues (preferably conservative amino acid residues) substituted, whereas such substituted amino acid residues may or may not be encoded by the genetic code, or (ii) a mutant protein having a substituted group in one or more amino acid residues, or (iii) a mutant protein formed by the fusion of the mature mutant protein to another compound, such as a compound that extends the half-life of the protein, such as polyethylene glycol, or (iv) a mutant protein formed by the fusion of an additional amino acid sequence to this mutant protein sequence (such as a secretory sequence or the sequence or protein used to purify this protein, or the fusion polypeptide formed with an antigenic IgG fragment). According to the teaching herein, these functional fragments, derivatives, and analogues belong to the common knowledge to those skilled in the art. In the present disclosure, conservatively substituted amino acids are preferably generated by amino acid substitutions according to Table 1.

TABLE 1

| | | Preferred |
|---|---|---|
| Initial residue | Representative substitution | substitution |
| Ala (A) | Val; Leu; Ile | Val |
| Arg (R) | Lys; Gln; Asn | Lys |
| Asn (N) | Gln; His; Lys; Arg | Gln |
| Asp (D) | Glu | Glu |
| Cys (C) | Ser | Ser |
| Gln (Q) | Asn | Asn |
| Glu (E) | Asp | Asp |
| Gly (G) | Pro; Ala | Ala |
| His (H) | Asn; Gln; Lys; Arg | Arg |
| Ile (I) | Leu; Val; Met; Ala; Phe | Leu |
| Leu (L) | Ile; Val; Met; Ala; Phe | Ile |
| Lys (K) | Arg; Gln; Asn | Arg |
| Met (M) | Leu; Phe; Ile | Leu |
| Phe (F) | Leu; Val; Ile; Ala; Tyr | Leu |
| Pro (P) | Ala | Ala |
| Ser (S) | Thr | Thr |
| Thr (T) | Ser | Ser |
| Trp (W) | Tyr; Phe | Tyr |
| Tyr (Y) | Trp; Phe; Thr; Ser | Phe |
| Val (V) | Ile; Leu; Met; Phe; Ala | Leu |

In addition, the mutant proteins of the present disclosure can also be modified. Modified (usually without changing the primary structure) forms include chemically derived forms of the protein such as acetylated or hydroxylated forms, in vivo or in vitro. Modifications also include glycosylation, such as those resulting from glycosylation modifications either in the synthesis and processing of the protein or in further processing steps. This modification can be accomplished by exposing the protein to enzymes (such as mammalian glycosylases or deglycosylases) for glycosylation. The modified forms also include sequences with phosphorylated amino acid residues (such as phosphotyrosine, phosphoserine, phosphothreonine). Mutant proteins that were modified to improve their antiproteolytic properties or optimize the solubilization properties were also included.

The term “polynucleotide encoding a mutant protein” may include a polynucleotide encoding a mutant protein of the present disclosure, or a polynucleotide that further includes additional coding and/or non-coding sequences.The present disclosure also relates to variants of the above-mentioned polynucleotides, wherein the variants encode fragments, analogues and derivatives of polypeptides or mutant proteins with the same amino acid sequences as the present disclosure. These nucleotide variants include degenerate variants, substitution variants, deletion variants, and insertion variants. As is known in the art, an allelic variant is a form of substitution of a nucleic acid, which may be a substitution, deletion, or insertion of one or more nucleotides but does not substantially alter the function of the protein it encodes.

The mutant proteins and polynucleotides of the present disclosure are preferably provided in an isolated form, more preferably, purified to homogeneity.

The nucleotide full-length sequence of the antibody of the description or fragment thereof can usually be obtained by PCR amplification method, recombination method or artificial synthesis method. Specifically, primers can be designed according to the nucleotide sequence disclosed herein, especially the open reading frame sequence, and a commercially available cDNA library or a cDNA library prepared by a conventional method known to those skilled in the art can be used as a template, and related sequences were amplified. When the sequence is long, it is often necessary to carry out two or more PCR amplifications, and then assemble the amplified fragments in correct order.

Once the relevant sequences are obtained, they can be obtained in large quantities by recombination. They are related sequences usually cloned into vectors, transferred into cells, and then isolated from the proliferated host cells by conventional methods.

In addition, synthetic methods are also available to synthesize the sequence in interest, especially for short fragments. Usually, fragments with very long sequence can be obtained by synthesizing several small fragments first and then connecting them.

At present, the DNA sequence encoding the protein (or fragment thereof, or derivative thereof) of the present disclosure can be obtained completely through chemical synthesis. This DNA sequence can then be introduced into various existing DNA molecules (or for example vectors) and cells known in the art. In addition, mutations can also be introduced into the protein sequence of the present disclosure through chemical synthesis.

The PCR technology to amplify DNA/RNA was preferably applied to obtain the polynucleotide of the present disclosure. In particular, when it is difficult to obtain a full-length cDNA from the library, the RACE method (RACE-cDNA Rapid amplification of cDNA ends) can be preferably used. The primers for PCR can be appropriately selected according to the sequence information of the present disclosure disclosed herein and can be synthesized by conventional methods. Amplified DNA/RNA fragments can be isolated and purified by conventional methods such as by gel electrophoresis.

### Expression Vectors and Host Cells

The present disclosure also relates to vectors containing the polynucleotides of the present disclosure, as well as host cells produced by genetic engineering methods using the vectors or coding sequences of the mutant proteins of the present disclosure, and methods for producing the polypeptides of the present disclosure through recombinant technology.

Through conventional recombinant DNA technology, the polynucleotide sequences of the present disclosure can be expressed or produced. Generally speaking, steps are as follows:
(1) transforming or transducing an appropriate host cell with a polynucleotide (including variants) encoding the mutant protein of the present disclosure, or with a recombinant expression vector containing the polynucleotide;
(2) culturing the host cells in a suitable medium;
(3) isolating and purifying proteins from the culture medium or cells.

In the present disclosure, the polynucleotide sequence encoding the mutant protein can be inserted into a recombinant expression vector. The term “recombinant expression vector” refers to bacterial plasmids, phages, yeast plasmids, plant cell viruses, mammalian cell viruses such as adenovirus, retrovirus or other vectors well known in the art. Any plasmid and vector can be used as long as it can replicate and be stable in the hosts. An important characteristic of an expression vector is that it usually contains an origin of replication, a promoter, a marker gene, and translation control elements.

Those skilled in the art are well known of methods for constructing expression vectors containing the present fusion protein coding sequences and suitable transcription/translation control signals. These include in vitro recombinant DNA techniques, DNA synthesis techniques, in vivo recombination techniques, etc. Said DNA sequence can be effectively ligated to an appropriate promoter in an expression vector to direct mRNA synthesis. Representative examples of these promoters are: the lac or trp promoter of E. coli; λ-phage PL promoter; eukaryotic promoters including the CMV immediate early promoter, the HSV thymidine kinase promoter, the early and late SV40 promoters, the LTR of retroviruses, and several other promoters known to control gene expression in prokaryotic or eukaryotic cells or their viruses. The expression vector also includes a ribosome binding site for translation initiation and a transcription terminator.

In addition, the expression vector preferably contains one or more selective marker genes to provide phenotypic traits for the selection of transformed host cells, such as dihydrofolate reductase for eukaryotic cell culture, neomycin resistance, and green Fluorescent protein (GFP), or for tetracycline or ampicillin resistance in E. coli.

Vectors comprising the appropriate DNA sequences described above, together with appropriate promoter or control sequences, can be used to transform appropriate host cells so that they can express the protein.

Host cells can be prokaryotic cells, such as bacterial cells; or lower eukaryotic cells, such as yeast cells; or higher eukaryotic cells, such as mammalian cells. Representative examples are: Escherichia coli, Streptomyces; bacterial cells of Salmonella typhimurium; fungal cells such as yeast, plant cells (for example, ginseng cells).

When the polynucleotides of the present disclosure are expressed in higher eukaryotic cells, transcription will be enhanced if an enhancer sequence is inserted into the vector. Enhancers are cis-acting elements of DNA, usually about 10 to 300 base pairs in length, acting on a promoter to enhance transcription of a gene. Illustrative examples include the SV40 enhancer of 100 to 270 base pairs on the late side of the origin of replication, the polyoma enhancer on the late side of the origin of replication, adenovirus enhancer, and other enhancers.

Those skilled in the art well know how to select appropriate vectors, promoters, enhancers, and host cells.

Transformation of host cells with recombinant DNA can be performed using conventional techniques known to those skilled in the art. When the host is a prokaryote such as Escherichia coli, competent cells that can absorb DNA can be harvested after the exponential growth period and treated with CaCl₂ method, the steps of which are well known in the art. Another method involves the use of MgCl₂. If necessary, the transformation can also be performed by electroporation. When the host is eukaryote, the following DNA transfection methods can be selected: calcium phosphate coprecipitation method, conventional mechanical methods such as microinjection, electroporation, liposome packaging, etc.

The obtained transformants can be cultured by conventional methods to express the polypeptide encoded by the gene of the description. According to the host cells used, the medium used in the culture can be selected from various conventional media. Culture is performed under conditions suitable for host cell growth. When the host cells grow to the appropriate cell density, the selected promoters are induced by appropriate methods (such as temperature conversion or chemical induction), and the cells are cultured for another period of time.

The polypeptide in the above method can be expressed inside the cell, on the cell membrane, or secreted outside the cell. If necessary, the recombinant protein can be separated and purified by various separation methods using its physical, chemical, and other characteristics. These methods are familiar to those skilled in the art. Examples of these methods include but are not limited to: conventional renaturation treatment, treatment with protein precipitant (salting-out method), centrifugation, permeation, ultra-treatment, ultra-centrifugation, molecular sieve chromatography (gel filtration), adsorption chromatography, ion exchange chromatography, high-performance liquid chromatography (HPLC) and other various liquid chromatography technologies and combinations of these methods.

### The Use

The CYP716A53v2 mutant of the present disclosure can specifically act on protopanaxadiol, adding a hydroxyl group to its C6 position to generate protopanaxatriol, and its catalytic activity is higher than that of wild-type CYP716A53v2. The catalysis is high-efficient catalysis, with its efficiency of at least more than 10% higher than that of the wild type, preferably at least more than 20% higher, more preferably at least more than 30% higher, such as more than 40% higher, more than 50% higher, more than 60% higher.

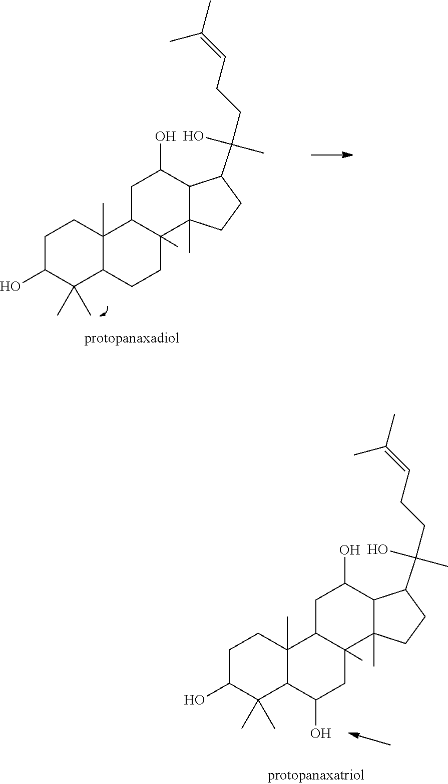 [SMILES: CC(C)=CCCC(C)(O)C1CCC2(C)C1C(O)CC1C3(C)CCC(O)C(C)(C)C3CCC12C ; CC(C)=CCCC(C)(O)C1CCC2(C)C1C(O)CC1C3(C)CCC(O)C(C)(C)C3C(O)CC12C]

After obtaining the CYP716A53v2 mutant of the present disclosure, according to the tips of the present disclosure, those skilled in the art can conveniently apply the mutant of the present disclosure to exert its catalytic effects on the substrate protopanaxadiol, and obtain significantly better technical effects than that of the wild type CYP716A53v2.

When used, especially in industrial production, the CYP716A53v2 mutant of the present disclosure or its derivative polypeptides can also be immobilized on a solid support to obtain an immobilized enzyme, which can be used for in vitro reaction with a substrate. The solid supports are, for example, microspheres, tubular bodies and other carriers made by inorganic substances. The preparation methods of immobilized enzymes are divided into two categories: physical methods and chemical methods. The physical methods comprise physical adsorption, embedding methods, etc. The chemical methods comprise bonding and cross-linking methods. The binding method is divided into ionic binding method and covalent binding method. The above-mentioned methods of immobilizing enzymes can all be applied in the present disclosure.

As an optional way, the CYP716A53v2 mutant of the present disclosure can be used for in vitro production, and the CYP716A53v2 mutant of the present disclosure can be produced on a large scale (it can be extract (including crude extract) or fermentation broth, or it can be isolated and purified product), and react in the presence of panaxadiol (as a substrate) to obtain panaxatriol product.

As another preferred mode of the present disclosure, the production is carried out by the method of biosynthesis. This typically includes: (1) providing an engineered cell with at least one characteristic selected from the group: an anabolic or production pathway comprising protopanaxatriol (PPT); (2) expressing the CYP716A53v2 mutant of the present disclosure in engineered cells described in (1), or replacing the CYP716A53v2 mutant of the present disclosure with the wild-type CYP716A53v2 in the metabolic pathway; and (3) culturing the engineered cells of (2) to produce a protopanaxatriol product. In a more preferred embodiment, the method further comprises the step of isolating and purifying the product from the culture of engineered cells.

When using the method of biosynthesis for production, as a preferred mode of the present disclosure, it also includes enhancing other compound metabolism pathways/production pathways in the protopanaxatriol (PPT) anabolic pathway of cells. It is also possible to provide more upstream substrates as precursors for the catalytic reactions of the present disclosure by enhancing the production of compounds in the upstream pathways of their anabolic pathways. It should be understood that other methods of enhancing the protopanaxatriol (PPT) anabolic pathway may also be encompassed by the present disclosure.

The CYP716A53v2 mutants of the present disclosure can also be used to prepare catalytic compositions. Those skilled in the art can determine an effective amount of the CYP716A53v2 mutant in the composition according to the actual use of the composition.

The CYP716A53v2 mutants, compositions comprising the mutants, the cells expressing the mutants, and other components of the present disclosure, can be included in kits for the convenience of scale-up applications or commercial applications. Preferably, the kit may further include a culture medium or culture components suitable for culturing the genetically engineered cells. Preferably, the kit also includes an instruction manual describing the method for biosynthesis, so as to guide those skilled in the art to carry out production in an appropriate method.

The disclosure if further illustrated by the specific examples described below. It should be understood that these examples are merely illustrative, and do not limit the scope of the present disclosure. The experimental methods without specifying the specific conditions in the following examples generally used the conventional conditions, such as those described in J. Sambrook, Molecular Cloning: A Laboratory Manual (3^{rd} ed. Science Press, 2002) or followed the manufacturer's recommendation.

The protein sequence of wild type CYP716A53v2
(SEQ ID NO: 1):
MDLFISSQLLLLLVFCLFLFWNFKPSSQNKLPPGKTGWPIIGETLEFIS
CGQKGNPEKFVTQRMNKYSPDVFTTSLAGEKMVVFCGASGNKFIFSNEN
KLVVSWWPPAISKILTATIPSVEKSKALRSLIVEFLKPEALHKFISVMD
RTTRQHFEDKWNGSTEVKAFAMSESLTFELACWLLFSINDPVQVQKLSH
LFEKVKAGLLSLPLNFPGTAFNRGIKAANLIRKELSVVIKQRRSDKLQT
RKDLLSHVMLSNGEGEKFFSEMDIADVVLNLLIASHDTTSSAMGSVVYF
LADHPHIYAKVLTEQMEIAKSKGAGELLSWEDIKRMKYSRNVINEAMRL
VPPSQGGFKVVTSKFSYANFIIPKGWKIFWSVYSTHKDPKYFKNPEEFD
PSRFEGDGPMPFTFIPFGGGPRMCPGSEFARLEVLIFMHHLVTNFKWEK
VFPNEKIIYTPFPFPENGLPIRLSPCTL

In the above sequence, the mutant positions are underlined in bold.

### Example 1. High-efficient Cytochrome P450 Mutant Protein Obtained by Random Mutation

(1) pUC57-synPPTS (a plasmid comprising the gene encoding CYP716A53v2) was used as a template. Primers SJ-F (SEQ ID NO: 2) and SJ-R (SEQ ID NO: 3) were used to perform error-prone PCR. The error-prone PCR was selected from Stratagene's GeneMorph II Random Mutagenesis Kit. The PCR program was: 95° C. for 2 min; 95° C. for 10 s, 55° C. for 15 s, 72° C. for 1 min 30 s, a total of 24 cycles; 72° C. for 10 min to 10° C. The amount of pUC57-synPPTS template used was 900 ng. PCR products were recovered by agarose gel electrophoresis to obtain CYP716A53v2 error-prone PCR products.

SJ-F: atggatttgtttatttcttc (SEQ ID NO: 2);

SJ-R: ttacaatgtacatggagaca (SEQ ID NO: 3).

(2) PCR reaction is carried out with the primers described in Table 2 and the template to amplify the target DNA fragment for strain construction. The PCR system is the high-fidelity PCR enzyme I-5™ 2xHigh-Fidelity Master Mixstandard system of Qingke Company. The PCR program was: 98° C. for 2 min; 98° C. for 10 s, 55° C. for 15 s, 72° C. for 1 min, a total of 30 cycles; 72° C. for 10 min to 10° C. The PCR products were recovered by agarose gel electrophoresis. The two fragment ends of the PCR product are respectively carried with about 70 bp homologous sequences to the adjacent two fragment ends by PCR primers, using for homologous recombination in Saccharomyces cerevisiae.

TABLE 2

| Forward primer/ | SEQ | | |
|---|---|---|---|
| Reverse primer | ID NO: | PCR template | PCR product |
| PPT-UP-F/ | 4/5 | Saccharomyces cerevisiae | PPT-UP |
| PPT-UP-R | | genome | |
| PPT-TEF1-F/ | 6/7 | Saccharomyces cerevisiae | PPT-TEF1 |
| PPT-TEF1-R | | genome | |
| PPT-PPTS-F/ | 8/9 | error-prone PCR product | PPT-PPTS |
| PPT-PPTS-R | | | |
| PPT-PRM9-F/ | 10/11 | Saccharomyces cerevisiae | PPT-PRM9 |
| PPT-PRM9-R | | genome | |
| PPT-KAN-F/ | 12/13 | pLKAN plasmid | PPT-KAN |
| PPT-KAN-R | | | |
| PPT-DN-F/ | 14/15 | Saccharomyces cerevisiae | PPT-DN |
| PPT-DN-R | | genome | |

PPT-UP-F:
(SEQ ID NO: 4)
cccaaagctaagagtcccat;
PPT-UP-R:
(SEQ ID NO: 5)
gtagaaacattttgaagctatggtgtgtgggggatcactctgctcttga
atggcgacag;
PPT-TEF1-F:
(SEQ ID NO: 6)
aacactggggcaataggctgtcgccattcaagagcagagtgatccccca
cacaccatag;
PPT-TEF1-R:
(SEQ ID NO: 7)
aacaataacaattgtgaagaaataaacaaatccattttgtaattaaaac
ttagattaga;
PPT-PPTS-F:
(SEQ ID NO: 8)
gaaagcatagcaatctaatctaagttttaattacaaaatggatttgttt
atttcttcac;
PPT-PPTS-R:
(SEQ ID NO: 9)
agtgtctcccgtcttctgtctaatgatgatgatgatgatgcaatgtaca
tggagacaat;
PPT-PRM9-F:
(SEQ ID NO: 10)
attgtctccatgtacattgcatcatcatcatcatcattagacagaagac
gggagacact;
PPT-PRM9-R:
(SEQ ID NO: 11)
ctgtcgattcgatactaacgccgccatccagtgtcgaattttcaacatc
gtattttccg;
PPT-KAN-F:
(SEQ ID NO: 12)
cattatgcaacgcttcggaaaatacgatgttgaaaattcgacactggat
ggcggcgtta;
PPT-KAN-R:
(SEQ ID NO: 13)
aattcaaaaaaaaaaagcgaatcttcccatgcctgttcagcgacatgga
ggcccagaat;
PPT-DN-F:
(SEQ ID NO: 14)
agactgtcaaggagggtattctgggcctccatgtcgctgaacaggcatg
ggaagattcg;
PPT-DN-R:
(SEQ ID NO: 15)
tctggtgaggatttacggtatg.

(3) After 100 ng of each of the above-mentioned product DNA fragments were mixed and transformed the competent strain ZW of Saccharomyces cerevisiae (Wang, P. P., et al., Cell Discovery, 2019.5(5)). After transformation, the strains were evenly spread on YPD+200 mg/L G418 antibiotic screening plates, and cultured at 30° C. for 48 h. All clones were picked with a toothpick and transferred to a 96-well plate, incubated at 30° C. with shaking for 24 hours, and transferred to a new 96-well plate at a ratio of 1:100 for 96 hours of fermentation.

Compound extraction: an equal volume of n-butanol solvent was added to the fermentation broth for 24 h extraction, then the upper organic phase was pipetted for HPLC detection of the yield and ratio of protopanaxadiol and protopanaxatriol in each transformant.

(4) After a lot of screening work, the inventors obtained a total of 4 clones with the protopanaxatriol yield PPT increased by 20% and the protopanaxatriol/protopanaxadiol ratio (PPT/PPD) increased by more than 20%, numbered SJ-1, SJ-2, SJ-3 and SJ-4, respectively. The cytochrome P450 fragment of each clone was obtained by PCR using the genomes of the above four clones as templates, and the cytochrome P450 fragment of each clone was obtained by using primers SJ-F and SJ-R, with the protein sequences of each mutant protein obtained by sequencing.

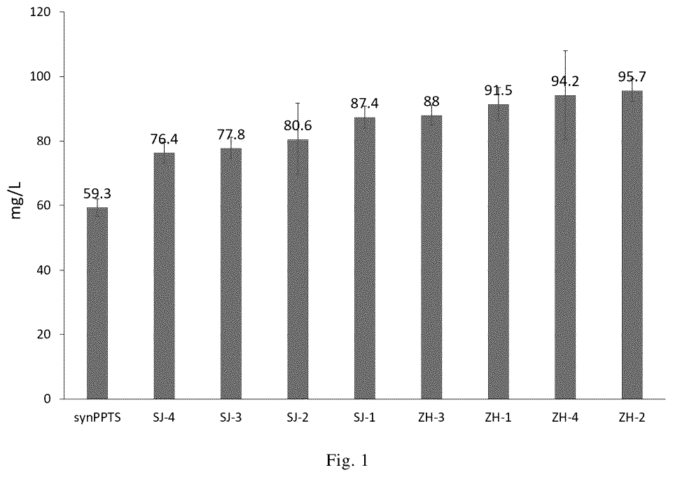

The information of protein sequence and PPT yield of each wild-type and mutant protein obtained above are shown in Table 3 and FIG. 1:

TABLE 3

| | Mutant | PPT yield | increased percent |
|---|---|---|---|
| Mutant | type | (mg/L) | of yield |
| CYP716A53v2 | Wild type | 59.3 | / |
| SJ-1 | F167V | 87.4 | 47.4% |
| SJ-2 | T451A | 80.6 | 35.9% |
| SJ-3 | I117S | 77.8 | 31.2% |
| SJ-4 | L208C | 76.4 | 28.8% |

### Example 2. By Integrating the Mutation Positions, a More Efficient CYP716A53v2 Mutant was Obtained

By the random mutation method in Example 1, a total of 4 positions that can improve activities were obtained: F167V, T451A, I117S, L208C.

On the basis of the wild-type CYP716A53v2 gene, a series of CYP716A53v2 mutant genes were obtained by various combinations of the above four mutant positions. Using the methods shown in (2) and (3) in Example 1, the combined mutant genes of CYP716A53v2 were transferred into ZW yeast competent cells respectively, and a series of corresponding strains were constructed for fermentation.

Fermentation method: 6 single clones of each mutant were picked into a 96-well plate, cultured with shaking at 30° C. for 24 hours, and transferred to a new 96-well plate at a ratio of 1:100 for fermentation for 96 hours (yeast itself can produce hydroxyl donors).

Compound extraction: an equal volume of n-butanol solvent was added to the fermentation broth for 24 h extraction, then the upper organic phase was pipetted for HPLC detection of the yield and ratio of protopanaxadiol and protopanaxatriol in each transformant.

TABLE 4

| | | PPT yield was |
|---|---|---|
| Mutant | Mutation position | increased by |
| synPPTS | None | 0 |
| ZH-1 | I117S, L208C | 54.3% |
| ZH-2 | I117S, T451A | 61.4% |
| ZH-3 | I117S, L208C, T451A | 48.4% |
| ZH-4 | I117S, L208C, F167V, T451A | 58.9% |

### Example 3. Efficient Heterologous Synthesis of Protopanaxatriol Using the Cytochrome P450 Mutant Protein

In this example, the cytochrome P450 mutant protein was used to efficiently synthesized heterologous protopanaxatriol. The specific method is as follows:

(1) PCR reaction is carried out with the primers described in Table 2 and the template to amplify the target DNA fragment for strain construction. The PCR system is the high-fidelity PCR enzyme I-5™ 2xHigh-Fidelity Master Mixstandard system of Qingke Company. The PCR program was: 98° C. for 2 min; 98° C. for 10 s, 55° C. for 15 s, 72° C. for 1 min, a total of 30 cycles; 72° C. for 10 min to 10° C. The PCR products were recovered by agarose gel electrophoresis. The two fragment ends of the PCR product are respectively carried with about 70 bp homologous sequences to the adjacent two fragment ends by PCR primers, using for homologous recombination in Saccharomyces cerevisiae.

All the above genes, homology arms and PCR fragments of selectable marker genes were mixed with 100 ng each, and then transformed into competent strain ZW of Saccharomyces cerevisiae to obtain a recombinant Saccharomyces cerevisiae strain PPT-WT strain for producing protopanaxatriol.

(2) Similarly, the mutant genes ZH-1, ZH-2, ZH-3, and ZH-4 were used as templates instead of the wild-type CYP716A53v2 gene. The above PCR was carried out to obtain each PCR fragment, then respectively transformed into competent strain ZW of Saccharomyces cerevisiae to obtain recombinant Saccharomyces cerevisiae strains with each mutant protein PPT-ZH-1, PPT-ZH-2, PPT-ZH-3, PPT-ZH-4 used for producing protopanaxatriol.

(3) Preparation of solid medium: Formulated medium: 1% yeast extract, 2% Bacto peptone, 2% D-glucose, 2% agar powder. Preparation of liquid medium: Formulated medium: Formulated medium: 1% yeast extract, 2% Bacto peptone, 2% D-glucose. (4) The recombinant strains of Saccharomyces cerevisiae PPT-ZH-1, PPT-ZH-2 , PPT-ZH-3, and PPT-ZH-4 were picked and streaked on a solid medium plate, and shaked overnight (30° C., 250 rpm, 16 h) in a tube containing 5 mL liquid medium; the cells were collected by centrifugation, transferred to a 50 mL conical flask with 10 mL liquid medium, with OD600 adjusted to 0.05, shaked and cultured for 4 days at 30° C., 250 rpm, to obtain the fermentation product. A parallel experiment was set up in this method for each strain of recombinant yeast at the same time.

(5) Extraction and detection of protopanaxatriol: 100 μL fermentation broth was pipetted from 10 mL fermentation broth. Fastprep was used to shake and lyse the yeast. An equal volume of n-butanol was added for extracting, then evaporated and dried the n-butanol under vacuum conditions. After dissolving with 100 μL methanol, the yield of the target product was detected by HPLC.

Protopanaxatriol obtained by each recombinant Saccharomyces cerevisiae strain above is shown in Table 5 and FIG. 1:

TABLE 5

| Strain | PPT yield mg/L |
|---|---|
| PPT-WT | 59.3 |
| PPT-ZH-1 | 91.5 |
| PPT-ZH-2 | 95.7 |
| PPT-ZH-3 | 88.0 |
| PPT-ZH-4 | 94.2 |

Each reference provided herein is incorporated by reference to the same extent as if each reference was individually incorporated by reference. In addition, it should be understood that based on the above teaching content of the disclosure, those skilled in the art can practice various changes or modifications to the disclosure, and these equivalent forms also fall within the scope of the appended claims.

1. A method for improving catalytic activities of cytochrome P450 CYP716A53v2, comprising: making mutation(s) in the amino acid sequence of cytochrome P450 CYP716A53v2, wherein the mutation(s) is mutated at position(s) 167, 451, 117 or 208 or a combination thereof, corresponding to wild-type CYP716A53v2 protein.

2. The method according to claim 1, wherein, position 167 is mutated to Val, position 451 is mutated to Asn, position 117 is mutated to Ser, or position 208 is mutated to Cys.

3. A cytochrome P450 CYP716A53v2 mutant, the mutant is:
(a) a mutant protein with an amino acid mutation corresponding to wild-type CYP716A53v2 at a position of 167, 451, 117 or 208 or a combination thereof;
(b) a protein derived from (a) and with the function of protein (a), formed by one or more amino acid residue substitution, deletion or addition of protein (a), but the residue corresponding to the position of 167, 451, 117 or 208 of wild-type CYP716A53v2 is as same as the corresponding residue of protein (a);
(c) a protein derived from (a) and with the function of protein (a), with more than 80% homology to the amino acid sequence of protein (a), but the residue corresponding to the position of 167, 451, 117 or 208 of wild-type CYP716A53v2 is as same as the corresponding residue of protein (a);
(d) a polypeptide derived from any one of the amino acid sequences of (a) to (c) with a tag or an enzyme-cleavage sequence added at N-terminus or C-terminus; or, a signal polypeptide or a secretory signal sequence fused at N-terminus.

4. The cytochrome P450 CYP716A53v2 mutant according to claim 3, wherein, position 167 is mutated to Val, position 451 is mutated to Asn, position 117 is mutated to Ser, or position 208 is mutated to Cys.

5. The cytochrome P450 CYP716A53v2 mutant according to claim 3, wherein, the cytochrome P450 CYP716A53v2 mutant comprises a protein corresponding to wild-type cytochrome P450 CYP716A53v2, where
(1) position 117 is mutated to Ser, position 451 is mutated to Ala;
(2) position 117 is mutated to Ser, position 208 is mutated to Cys, position 167 is mutated to Val, position 451 is mutated to Ala;
(3) position 117 is mutated to Ser, position 208 is mutated to Cys;
(4) position 117 is mutated to Ser, position 208 is mutated to Cys, position 451 is mutated to Ala;
(5) position 167 is mutated to Val;
(6) position 451 is mutated to Ala;
(7) position 117 is mutated to Ser; or
(8) position 208 is mutated to Cys.

6. An isolated polynucleotide, wherein, the nucleic acid encodes the cytochrome P450 CYP716A53v2 mutant according to claim 3.

7. A vector comprising the polynucleotide according to claim 6.

8. A genetically engineered host cell, comprising the vector according to claim 7.

9. The host cell according to claim 8, wherein the host cell is a eukaryotic cell or a prokaryotic cell.

10. A method for preparing a cytochrome P450 CYP716A53v2 mutant according to claim 3, comprising:
(i) culturing a host cell comprising a nucleic acid encoding said cytochrome P450 CYP716A53v2 mutant;
(ii) collecting a culture comprising said cytochrome P450 CYP716A53v2 mutant;
(iii) separating said cytochrome P450 CYP716A53v2 mutant from the culture.

11. A composition for catalyzing protopanaxadiol to generate protopanaxatriol, comprising an effective amount of: the cytochrome P450 CYP716A53v2 mutant according to claim 3; or, a host cell comprising a nucleic acid encoding the cytochrome P450 CYP716A53v2 mutant; and
food-acceptable or industrially acceptable carrier.

12. A method for producing a composition for catalyzing protopanaxadiol to generate protopanaxatriol, comprising mixing an effective amount of the cytochrome P450 CYP716A53v2 mutant according to claim 3 with a food-acceptable or industrially acceptable carrier.

13. A method for catalyzing protopanaxadiol to generate protopanaxatriol, wherein the method comprises: utilizing the cytochrome P450 CYP716A53v2 mutant according to claim 3 to generate protopanaxatriol by adding a hydroxyl group at the C6 position of protopanaxadiol.

14. A kit for catalyzing protopanaxadiol to generate protopanaxatriol, comprising:
the cytochrome P450 CYP716A53v2 mutant or the combination of mutants thereof according to claim 3; a host cell comprising a nucleic acid encoding said cytochrome P450 CYP716A53v2 mutant; or
the composition comprising an effective amount of said cytochrome P450 CYP716A53v2 mutant.
